(19) United States
(12) Patent Application Publication
(10) Pub. No.: US 2010/0247581 A1
(43) Pub. Date: Sep. 30, 2010

(54) EDIBLE PRODUCT HAVING AN IMMUNOSTIMULATING EFFECT

(76) Inventors: Salomon Leendert Abrahamse, Vlaardingen (NL); Johanna Alida Van Adrichem, Vlaardingen (NL); Wilhelmina Martina Blom, Vlaardingen (NL); Monique Cecilia D. van der Burg-Koorevaar, Vlaardingen (NL); Jean Hypolites Koek, Vlaardingen (NL)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/745,992

(22) PCT Filed: Nov. 13, 2008
(86) PCT No.: PCT/EP2008/065445

§ 371 Date: Jun. 3, 2010

(30) Foreign Application Priority Data
Dec. 7, 2007 (EP) 07122634.4

(51) Int. Cl. A61K 31/715; C08B 37/00

(52) U.S. Cl. 424/278.1; 536/123.1

(57) ABSTRACT

There is provided an edible product having an immunostimulating effect, said product comprising immunostimulating polysaccharides obtainable from plants of the Asclepiadoideae subfamily. Also provided is a process for preparing such an edible product and a composition comprising from 0.0001 to 25% by weight of polysaccharides having an immunostimulating effect.

### TECHNICAL FIELD

The present invention relates to an edible product. More in particular, it relates to an edible product having an immunostimulating effect, especially an edible product comprising immunostimulating polysaccharides obtainable from plants of the Asclepiadoideae subfamily.

### BACKGROUND ART

Some edible products or food products are known to have immunostimulating properties. For example, WO-A-2007/054208 (Unilever) discloses edible products containing probiotic bacteria in an amount of at least 10³ bacteria per gram and at least 0.5 mg/g of ginseng polysaccharides containing at least 2 monosaccharide units for restoring or maintaining immune function.

U.S. Pat. No. 6,432,454 (C.V. Technologies) discloses a process of making fractions from North American ginseng (Panax quinquefolium) and compositions containing these fractions, which may be used to stimulate the production of cytokines and/or antibodies, or as therapeutics targeted at conditions characterized by low immunity such as the common cold, influenza, chronic fatigue syndrome, AIDS and cancer. Ginseng, however, is an expensive ingredient.

There is a constant need for new or alternative food products having such immunostimulating properties. It is therefore an object of the present invention to provide such edible products. It is a further object of the invention to provide a process for the preparation of such food products having such immunostimulating properties.

It was surprisingly found that the object can be achieved by the edible product of the invention, which comprises immunostimulating polysaccharides obtainable from plants of the Asclepiadoideae subfamily.

The Asclepiadaceae is a former plant family now treated as a subfamily (subfamily Asclepiadoideae) in the Apocynaceae family. They form a group of perennial herbs, twining shrubs, lianas, or rarely trees but notably also contain a significant number of leafless stem succulents, all belonging to the order Gentianales.

The subfamily comprises the tribe Stapelieae, to which the genus Hoodia belongs, a succulent plant found in the Kalahari dessert of South Africa. Although this plant has a spiny appearance similar to cacti, they are unrelated to the cactus family. Hoodia belongs to a genus of 13 species in the flowering plant family Apocynaceae, under the subfamily Asclepiadoideae. Steroidal glycosides from Hoodia have been reported to be active constituents of this plant acting as an appetite-suppressant. WO-A-98/46243 discloses that these plants contain steroidal glycosides having the formula 1:

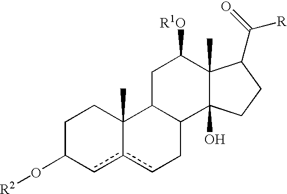

wherein
R=alkyl;
R¹=H, alkyl, tiglyol, benzoyl or any other organic ester group;
R²=H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

WO-A-98/46243 also discloses a process to extract the steroidal glycoside having the formula 1 from plants of the Asclepiadoideae subfamily, involving treating plant material with a solvent to extract a fraction having appetite suppressant activity, separating the extraction solution from the rest of the plant material, removing the solvent from the extraction solution and recovering the extract. The solvents specifically mentioned to perform the extraction are one or more of methylene chloride (dichloromethane), water, methanol, hexane, ethyl acetate or mixtures thereof.

A recent interest in the field of functional food ingredients is the use of immunomodulators for enhancing host defence responses. An important part of the host defence response is the innate immune system. The innate arm of the immune system is a rapidly activated first line of defence against pathogens. It involves amongst others phagocytic and natural killer (NK) cells. Phagocytic cells such as neutrophils, monocytes and macrophages can generate reactive oxygen species (ROS) to kill pathogens such as fungi, bacteria and virus-infected cells. NK cells can kill target cells that have lost or express insufficient amounts of MHC class I, a frequent event in tumor- or virus-infected cells.

Plant, algae and mushroom derived polysaccharides can exhibit a number of beneficial therapeutic properties, including immunostimulation. Polysaccharides from for example Echinacea purpurea, Plantago ovata, Panax ginseng, Panax quinquefolius, buplureum falcatum and Poria cocos have been described to affect macrophage function such as activation of phagocytic activity, increase in ROS and nitric oxide production and enhanced secretion of cytokines and chemokines. However, the immune stimulating effect of polysaccharides from plants of the Asclepiadoideae subfamily or of Hoodia Gordonii has never been disclosed.

### SUMMARY OF THE INVENTION

According to a first aspect of the present invention, there is provided an edible product having an immunostimulating effect, said product comprising immunostimulating polysaccharides obtainable from plants of the Asclepiadoideae subfamily.

According to a second aspect of the invention, there is provided a process for preparing the edible product according to the invention.

According to a third aspect of the invention, there is provided a composition comprising from 0.0001 to 25% by weight of polysaccharides obtainable from plants of the Asclepiadoideae subfamlily and having an immunostimulating effect and having an average degree of polymerization >3.

### DETAILED DESCRIPTION OF THE INVENTION

According to a first aspect, the present invention relates to an edible product comprising polysaccharides which are responsible for the immunostimulating effect. The immunostimulating polysaccharides used in the present product may be derived from plants belonging to the Asclepiadoideae subfamily.

Preferably, the immunostimulating polysaccharides are derived from plants of the tribe Stapelieae. More preferably, they are derived from the genus Hoodia, which comprises 13 species.

It is especially preferred if the plant is selected from the species Trichocaulon piliferum, Trichocaulon officinale, Hoodia currorii, Hoodia gordonii, Hoodia lugardii and mixtures thereof. Hoodia gordonii is especially preferred.

The immunostimulating polysaccharides may be isolated from the plants of a member of the Asclepiadoideae subfamily by cutting up the plant, extracting the cut material (for instance with MeOH:water (90:10% w/w) and preparing a hot-water extract from the insoluble material of the previous step. A polysaccharide enriched extract may be prepared using additional washing steps with e.g. ethanol. Preferably the extract is obtained from plants of the Hoodia genus. It is especially preferred if the immunostimulating polysaccharides are obtainable from plants of the species Hoodia gordonii.

The immunostimulating polysaccharides can be characterised by their average degree of polymerization which is >3, preferably >5, more preferably >7, up to 1,000.

The edible product according to the present invention may take any physical form. In particular, it may be a liquid or a spreadable, spoonable solid or a food supplement.

Preferably the product is a liquid product. The edible product may suitably take the form of e.g. a soup, a beverage, a spread, a dressing, a dessert or a mayonnaise. More preferably, the edible product is a beverage, a dessert or a spread. More preferably, the edible product is a beverage or a spread, especially a spread in the form of an oil-in-water emulsion. The term “spread” as used herein encompasses spreadable products such as margarine, spreadable cheese based products and processed cheese. Most preferably, the present product is a beverage. Such a beverage typically contains at least 60 wt. % water and 0-20 wt. % of dispersed fat. Preferably, such beverage contains at least 70 wt. % water and 0-10 wt. % of dispersed fat.

The edible product according to the present invention preferably includes one or more additional conventional nutrients, vitamins and minerals to deliver healthy nutrition, despite the immune stimulation. Suitable vitamins and minerals, include but are not limited to Vitamin A, Vitamin D, Vitamin E, Vitamin C, Thiamin, Riboflavin, Niacin, Vitamin B6, folate, Vitamin B12, Biotin, Pantothenic acid, Calcium, Phosphorous, Potassium, Iron, Zinc, Copper, Iodine, Selenium, Sodium, Magnesium, Manganese, molybdenum, vitamin K, chromium and mixtures thereof. The preferred ingredients to deliver vitamins and minerals include but are not limited to potassium phosphate, calcium phosphate, magnesium oxide, magnesium phosphate, ascorbic acid, sodium ascorbate, vitamin E acetate, niacinamide, ferric orthophosphate, calcium pantothenate, zinc oxide, zinc gluconate, vitamin A palmitate, pyridoxine hydrochloride, riboflavin, thiamin mononitrate, biotin, folic acid, chromium chloride, potassium iodide, sodium molybdate, sodium selenate, phytonadone (vitamin K), cholecalciferol (vitamin D3), cyanocobalamin (vitamin b12), manganese sulfate and mixtures thereof. Preferably, the inventive product contains at least 10% or more of the recommended daily amount (“RDA”) of the vitamins and minerals.

The inventive products may further include meat, fish, meat and fish extracts, fruit, dried fruit, fruit concentrates, fruit extracts, fruit juices, tea (e.g. green tea), vegetables, vegetable extracts and concentrates, nuts, nut extracts, chocolate, bread, vinegar, salt, pepper, cocoa powder, herbs (e.g. parsley), herb extracts, spices (e.g. cinnamon), spice extracts, emulsifiers, acidity regulators (e.g. phosphoric, malic, citric, tartaric acids and salts thereof), flavonoids, preservatives (e.g. lactic acid, EDTA, tocopherols, sodium benzoate), colors (e.g. beta carotene, lycopene, caramel, carmine red), fibers (e.g. soy), leavening agents (e.g., sodium bicarbonate), pectin, citric acid, yeast, salt, glycerin, and mixtures thereof.

Optionally, the edible product may further comprise one or more appetite suppressing steroidal glycosides having the formula 1:

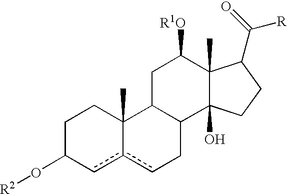

wherein
R=alkyl;
R¹=H, alkyl, tiglyol, benzoyl or any other organic ester group;
R²=H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

By the term “immunostimulating” as used herein is meant that the activity or capacity of the immune system to defend itself against pathogens such as fungi, bacteria, viruses, protozoa, parasites or proteins is increased. Several assays can be used to identify components that could modify immunity. The present inventors chose the use of phagocytic and natural killer (NK) cells to aid the identification of immunostimulating compounds as these cells are part of the innate immune system, which is a rapidly activated non-specific first line of defence against pathogens.

Phagocytic cells such as neutrophils, monocytes and macrophages can generate reactive oxygen species (ROS) to kill pathogens such as fungi and bacteria. The effect of ingredients on phagocytosis activity can be measured ex vivo with fresh blood of healthy human volunteers after incubation with FITC-labelled E. coli bacteria. The percentage of phagocytosing cells in the granulocyte population can be determined by flow cytometry. The results are typically normalized to the effect of lipopolysaccharide (LPS), which is a well known potent immunostimulating reference compound. In the present invention a normalized % phagocytosing granulocytes >40% is regarded as a significant immune stimulating effect.

NK cells can kill target cells that have lost or express insufficient amounts of MHC class I, a frequent event in tumor- or virus-infected cells. The effect of ingredients on NK cell activity can ex vivo be measured with peripheral blood mononuclear cells (PBMC) isolated from fresh blood of healthy human volunteers. After pre-incubation of the PBMCs with the ingredient, pre-labelled K562 target cells are usually added and after subsequent incubation, propidium iodide can be added for detection of dead cells. The percentage of dead target cells can be determined with flow cytometry. The results are typically normalized to the effect of interleukin-2 (IL-2), which is a well known potent NK cell stimulating reference compound. In the present invention a normalized % NK cell activity >17% is regarded as a significant immune stimulating effect.

According to a second aspect of the invention, the edible product according to the invention is prepared by adding the immunostimulating polysaccharides to a food product, e.g. during the production process.

A further aspect of the present invention is a process to obtain the polysaccharides having an immunostimulating effect. Plant based polysaccharides consist of large insoluble polymers, like cell wall components, small soluble oligosaccharides, like monomers (e.g. glucose) and dimers (e.g. cellobiose), and large soluble polysaccharides. Especially from the latter an immunomodulating response may be expected as they are large enough to provoke a reaction from the immune system and solubility is a requirement for interaction. The polysaccharide containing extract may be prepared as described above. In order to enrich plant based material in soluble polysaccharides, the small oligosaccharides may have to be removed. This is usually done (Shiomi, N., 1992. New Phytologist 122, pp. 421-432) by a repeated warm alcohol (85%) wash step as the small oligosaccharides have some solubility in the aqueous alcohol while the polymers are insoluble. Subsequently a hot water extraction is applied to the residue to isolate the water soluble polymer and separate it from insoluble polysaccharides. In this step also other water insoluble components are removed. In order to check the success of carbohydrate isolation an overall content of carbohydrates is determined using the Dubois method (Dubois M et al, (1956) Colorimetric method for determination of sugars and related substances, Analytical chemistry, 28(3), 350-356). A first rough insight in the success of removal of small oligosaccharides is obtained by the average degree of polymerization which is determined by comparing the analysis result on carbohydrate reducing end groups (DNSA method) with the total carbohydrate content determined by the Dubois method. Successful removal of small oligosaccharides (e.g. mono and disaccharides) would give a high average DP value (e.g. at least higher than 2). A more accurate way is to determine the molecular weight distribution of the enriched extract by size exclusion chromatography.

A further aspect of the invention is a composition comprising from 0.0001 to 25% by weight of polysaccharides obtainable from obtainable from plants of the Asclepiadoideae subfamily and having an immunostimulating effect, said polysaccharides having an average degree of polymerization >3, preferably >5, more preferably >7 up to 1,000.

This invention will now be described in more detail by means of the following Examples.

### Example 1

### Extraction Procedure to Obtain an Edible Composition Comprising Immunostimulating Polysaccharides from Hoodia Gordonii (A) and a Polysaccharide-Enriched Extract of Hoodia Gordonii (B)

Hoodia Gordonii plants were cut and the cut-material was extracted with MeOH:water (90:10% w/w). 0.2 g of the MeOH insoluble plant material was incubated for 5 minutes in 10 ml of boiling Limulus Amebocyte Lysate (LAL) water (Cambrex, US) and subsequently centrifuged at 3,000 g for 45 min at 4° C. The supernatant which contained polysaccharides from Hoodia Gordonii (A) was filtered over a 0.2 μm filter, divided in small portions and stored at −20° C., until use in the immune assays.

The polysaccharide-enriched extract of Hoodia Gordonii (B) was obtained as follows: 25 g of the MeOH insoluble plant material was washed 2 times with 200 ml of 85% ethanol (VWR Prolabo) in water for 2.5 hours at 80° C. and 1 time with 200 ml of 85% ethanol in water for 1.5 hours at 80° C. After decanting the ethanol, the pellet was dried overnight in a fuming cabinet. The polysaccharides were extracted by adding 200 ml of MilliQ water and boiling for 3 hours. After centrifugation at 2,000 g for 20 min at room temperature (RT), the pellet was re-suspended in 200 ml of MilliQ water and boiled again for 3 hours. The supernatant of the first and second extraction were collected, lyophilized and stored at RT. From the polysaccharide enriched lyophilized Hoodia Gordonii powder a 2% (m/m) suspension in LAL water was made and autoclaved for 15 minutes at 121° C. The suspension was centrifuged at 2000 g for 30 minutes at RT. The supernatant was filtered through a 0.2 μm filter, divided in small portions and stored at −20° C., until use in the immune assays.

### Example 2

### Analytical Characterization of the Edible Composition Comprising Immunostimulating Polysaccharides from Hoodia Gordonii (A) and a Polysaccharide-Enriched Extract of Hoodia Gordonii (B)

### Quantification of Carbohydrates

The total amount of carbohydrates in the sample was measured according to the method of Dubois (vide supra). 50 μl of test sample or standard curve sample (0-0.3 g/l glucose) was mixed with 20 μl of 6 g/l resorcinol (Aldrich) and 90 μl pure H₂SO₄ p.a. (Merck). After an incubation of 20 minutes at 80° C. the extinction was measured at 450 nm. The amount of carbohydrates was calculated via linear regression of the calibration curve with glucose.

### Determination of the Monosaccharide Composition

A 10 g/l solution of an edible composition comprising polysaccharides from Hoodia Gordonii (a) and a polysaccharide-enriched extract of Hoodia Gordonii (b) were hydrolyzed in 2 M HCL to obtain monosaccharides. The hydrolysis was done by addition of 0.2 ml 37% hydrochloric acid to 1 ml solution giving a final concentration of 2 M HCl, followed by thoroughly mixing and an incubation of 6 hours at 95° C. in a pre-heated water bath. After this incubation, the solution was cooled down to RT and centrifuged for 10 minutes at 15600×g. The pH of the supernatant was adjusted to a pH between 3 and 7 with 10 M NaOH, filter-sterilized (0.45 μm) and 0.5 ml was placed in a 1 ml HPLC vial for analysis (LC-10AT, Shimadzu, Japan). Both monosaccharide samples (10 μl) were injected. For calibration, monosaccharides (10 μl solutions of 1 g/l concentrations) were injected (see table 2 list of monosaccharides used for calibration, all were purchased at Sigma-Aldrich) using an auto injector SIL-10AD, Shimadzu, Japan. Sulphuric acid (5 mM, pH 2.0) was used as an eluent and an Aminex HPX-87H (300×7.8 mm) column was used at a temperature of 65° C. and a flow-rate of 0.6 ml/min. Dual determination of refractive index (RID-10A, Shimadzu, Japan) and UV 220 nm and 280 nm (SPD-10A, Shimadzu, Japan) was used. Based on the calibration with the monosaccharide's the monosaccharide composition present in the hydrolyzed samples was determined.

### Determination of the Degree of Polymerization

The molecular amount of polysaccharides in the samples was measured according to the method of Bernfeld (Bernfeld et al (1955) Amylase alpha and beta. Methods in Enzymology, 1, 149-158). A 3,5-dinitrosalicylic acid (DNSA) reagent was made by dissolving one gram DNSA in 20 ml 2 M NaOH and 50 ml water at 60° C. After obtaining a clear solution, 30 g potassium sodium tartrate was added and the volume was adjusted to 100 ml. 150 μl of test sample or standard curve sample (0-25 mmol/l glucose) was mixed with 150 μl DNSA reagent. Three times 50 μl of the sample DNSA reagent mixture was added to a 96-well plate. After heating for 30 minutes at 80° C. and cooling to RT, 100 μl of water was added and the absorbance at 540 nm was measured against a blank. The molecular amount of polysaccharides was calculated via linear regression of the calibration curve with glucose.

By dividing the amount of carbohydrates (g/kg sample) with the molecular amount (mol/kg sample), the average molecular weight (g/mol) was determined.

### Determination of the Molecular Weight Distribution

The distribution of the molecular weight was done with preparative size exclusion chromatography performed with Äcta explorer (GE Healthcare, Sweden) with a P900 pump and a UV900 detector. The Sephacryl S200 HR was packed in an XK100/1.6 column according to the manufacturers instructions (GE Healthcare note 52-2086-00), using D-PBS without CaCl₂ and MgCl₂ (GIBCO BRL) as a buffer, pH=7.0. The column was equilibrated with D-PBS buffer without CaCl₂ and MgCl₂ at a flow rate of 1 ml/min. 2 ml samples or standard was loaded at a flow rate of 0.5 ml/min and the isocratic elution was done at 0.5 ml/min. For the dextran standards of 80 kD, 50 kD, 25 kD, 12.5 kD, 5 kD and 1 kD, a concentration of 1 g/l was used. For the polysaccharide-enriched extract of Hoodia Gordonii (B) 5 g/l in buffer was used. Fractions of 4.8 ml were collected between 65 ml and 180 ml during elution of the samples. Of the fractions the amount of carbohydrates was measured according to the method of Dubois (vide supra).

Characterization of an Edible Composition Comprising Polysaccharides from Hoodia Gordonii (A) or a Polysaccharide-Enriched Extract of Hoodia Gordonii (B)

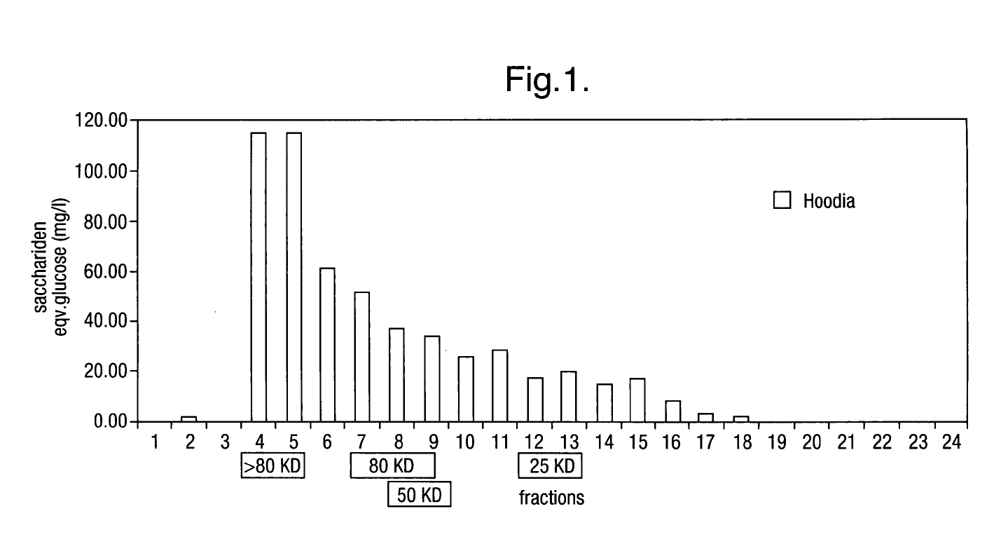

The total amount of carbohydrates, the monosaccharide composition, the degree of polymerization and the protein content of an edible composition comprising polysaccharides (PS) of Hoodia Gordonii (A) and a polysaccharide-enriched extract of Hoodia Gordonii (B) are shown in table 1. The molecular weight distribution of the polysaccharide-enriched extract of Hoodia Gordonii (B) is shown in FIG. 1. The composition of extract A is characterized by carbohydrates in the range between 8 and 10%, different monosaccharides and total protein in the range between 7 and 8%. The composition of extract B, the PS-enriched extract of Hoodia Gordonii is characterized by carbohydrates in the range between 37 and 42%, similar monosaccharides as extract A but in different quantities, total protein in the range between 19 and 21% and an average degree of polymerization >6.

TABLE 1
Characterization of an edible composition comprising immunostimulating
polysaccharides from Hoodia Gordonii (A) and a polysaccharide-
enriched extract of Hoodia Gordonii (B).

| | Edible composition | Polysaccharide- |
|---|---|---|
| | comprising PS from | enriched extract of |
| | Hoodia Gordonii (A) | Hoodia Gordonii (B) |
| Carbohydrate (CH) | 8-10 | 37-42 |
| (weight % dry matter) | | |

Monosaccharide composition (% of total CH)

| | | |
|---|---|---|
| Glucuronic acid | 8 | 13 |
| Galacturonic acid | 21 | 23 |
| Glucose | 21 | 22 |
| Galactose/Xylose/ | 29 | 29 |
| Fructose | | |
| Rhamnose | 4 | 4 |
| Fucose | 1-2 | 1-2 |
| Arabinose | 5 | 5 |
| unknown | 9 | |
| Degree of | n.d. | >6 |
| polymerization | | |
| Protein | 7-8 | 19-21 |
| (weight % dry matter) | | |

### Example 3

### Natural Killer (NK) Cell Stimulating Effect of an Edible Composition Comprising Polysaccharides of Hoodia Gordonii

### Determination of Natural Killer (NK) Cell Activity

Fresh blood was obtained from healthy volunteers in sodium heparin tubes and peripheral blood mononuclear cells (PBMC) were isolated from the blood by density gradient centrifugation using Ficoll-Paque. PBMC were counted and resuspended in tissue culture medium RPMI 1640 Complete at a concentration of 5×10⁶ cells/ml and stored at 4° C. until use as effector cells in the flow cytometric cytotoxicity assay. The erythromyelocytic leukemia cell line K562, a NK sensitive cell line, was used as target cells. K562 cells were washed with PBS, counted and resuspended in PBS at a concentration of 5×10⁶ cells/ml before labeling with the dye carboxyfluorescein diacetate succinimidyl ester (CFDA-SE) at 1 μg/ml for 30 min at 5% CO₂. 37° C. After labeling, cells were washed with PBS and resuspended in RPMI Complete at a concentration of 10×10⁴ cells/ml and stored at 4° C. in the dark until use.

PBMCs were pre-incubated with the ingredient in triplicate for 30 minutes at 37° C. (5% CO₂). Control incubations consisted of PBS (=basal level of NK cell activity) or 800 IU/ml interleukin-2 (IL-2) (=positive control sample) (mean ±sd in triplicate). After pre-incubation with the ingredient, target cells were added (ratio effector:target cells=25:1) and incubated for 120 minutes at 37° C. (5% CO₂). After incubation, cells were placed on ice for 1-5 minutes after addition of propidium iodide for detection of dead cells. Natural killer cell activity was measured on the Coulter FC500 MPL flow cytometer (Beckman Coulter, Miami, Fla., USA). Data of at least 1000 target cells were collected and analyzed using the EXPO 32 program. The percentage of dead target cells was determined. The results were normalized to the effect of IL-2.

Modulation of NK Cell Activity by Polysaccharides from Hoodia Gordonii

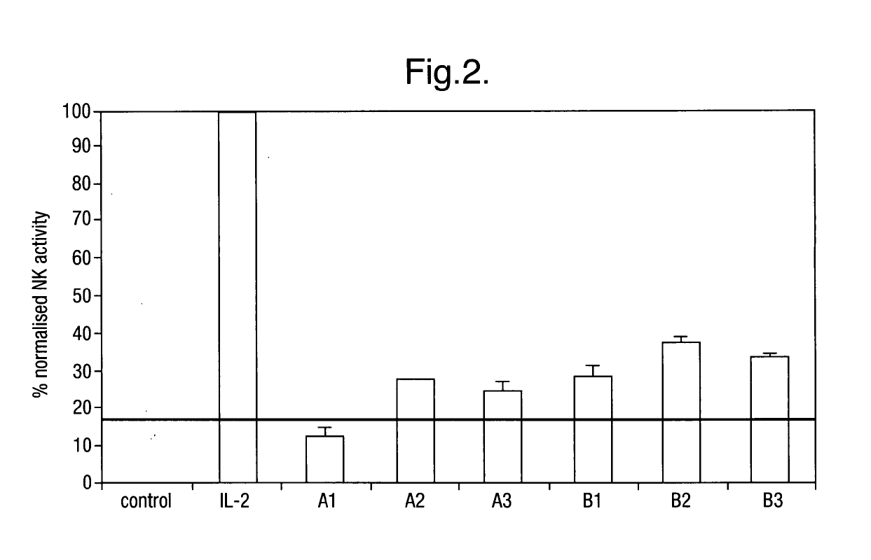

An edible composition comprising polysaccharides from Hoodia Gordonii (A) and a polysaccharide-enriched extract of Hoodia Gordonii (B) were tested at three different concentrations in the NK cell activity assay (1=0.4 μg/ml; 2=4 μg/ml; 3=40 μg/ml). The results of this experiment are shown in FIG. 2. It demonstrates the NK cell stimulating activity of an edible composition comprising polysaccharides of Hoodia Gordonii (A) at a concentration of 0.4 (A1)-4 (A2)-40 (A3) μg/ml and a polysaccharide-enriched extract of Hoodia Gordonii (B) at a concentration of 0.4 (B1)-4 (B2)-40 (B3) μg/ml. The NK cell activity is reported as percentage of maximal stimulation by interleukin-2, whereby a % normalized NK cell activity of >17% can be regarded as an immune stimulating effect (=arbitrary threshold). Results represent means and the standard deviation of one experiment with each sample performed in duplicate. The results show that an edible composition comprising polysaccharides from Hoodia Gordonii (A) stimulates NK cell activity of PBMC isolated from human blood samples at a concentration of 4 and 40 μg/ml. A polysaccharide-enriched extract of Hoodia Gordonii (B) is already effective at a concentration of 0.4 μg/ml.

### Example 4

### Phagocytosis Stimulating Effect of an Edible Composition Comprising Polysaccharides of Hoodia Gordonii

### Determination of Phagocytosis Activity

Phagocytosis activity was measured with the Phagotest® kit of Orpegen Pharma (Heidelberg, Germany) using an adjusted protocol. In more detail:

Fresh blood was obtained from healthy human volunteers in sodium heparin vacutainers (BD biosciences). 30 ul of whole blood and 5 ul of the ingredient were pre-incubated in duplicate for 30 minutes in a polypropylene 96-well plate at 37° C. in a water bath. Control incubations consisted of PBS (=basal phagocytosis activity) or 100 ng/mL E. coli-lipopolysaccharide (LPS) (=positive control sample) (mean±sd in triplicate). After the pre-incubation step, 10 μl of FITC-labeled E. coli (white blood cell to E. coli ratio of 25:1) was added. This incubation at 37° C. was stopped after 6.5 minutes by adding 50 ul of ice-cold quencher solution. The cells were washed three times by adding 230 μl of ice-cold wash-buffer and centrifugation for 3 min at 300 g (4° C.). The erythrocytes were lysed by addition of 290 μl of lysis buffer. After incubation in the dark for 20 minutes at room temperature, the cells were centrifuged for 5 min at 300 g (4° C.). Cells were resuspended in 150 μl of wash-buffer and stained with propidium iodide. Analysis was performed by flow cytometry (Coulter FC500 MPL flow cytometer, Beckman Coulter Nederland BV, Mijdrecht). Leukocytes were gated into monocyte and granulocyte populations according to the FSC/SSC profile. The percentage of phagocytosing cells in the granulocyte population was determined. The results were normalized to the effect of lipopolysaccharide (LPS).

Modulation of Phagocytosis Activity by Polysaccharides from Hoodia Gordonii

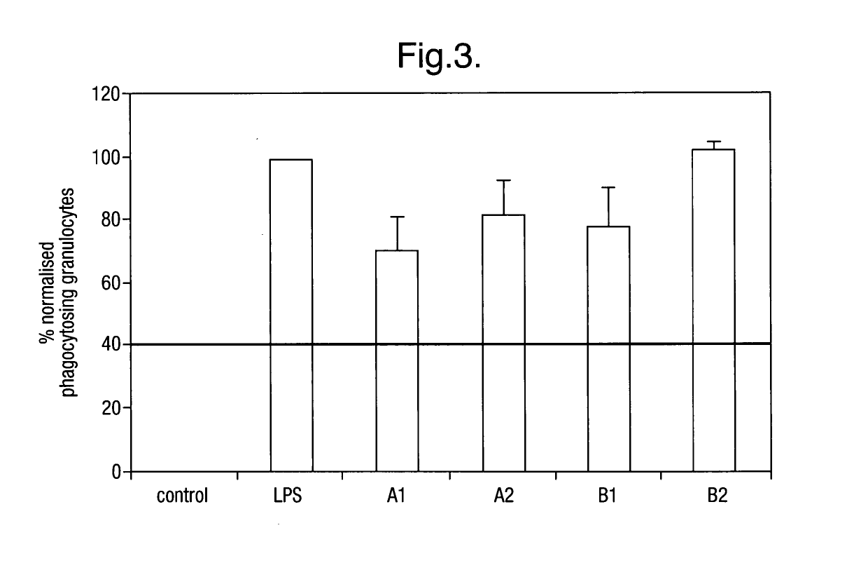

An edible composition comprising polysaccharides from Hoodia Gordonii (A) and a polysaccharide-enriched extract of Hoodia Gordonii (B) were tested at two different concentrations in the phagocytosis activity assay (1=29 μg/ml; 2=290 μg/ml). The results of this experiment are shown in FIG. 3. It demonstrates the phagocytosis stimulating activity of an edible composition comprising polysaccharides of Hoodia Gordonii (A) at a concentration of 29 (A1)-290 (A2) μg/ml and a polysaccharide-enriched extract of Hoodia Gordonii (B) at a concentration of 29 (B1)-290 (B2) μg/ml. The percentage of phagocytosing granulocytes is reported as percentage of maximal stimulation by LPS, whereby a % normalized phagocytosing granulocytes of >40% can be regarded as an immune stimulating effect (=arbitrary threshold). Results represent means and the standard deviation of one experiment with each sample performed in duplicate. The results show that an edible composition comprising polysaccharides from Hoodia Gordonii (A) and a polysaccharide-enriched extract of Hoodia Gordonii (B) both stimulate phagocytosis activity of granulocytes isolated from fresh human blood samples at a concentration of 29 and 290 μg/ml.

### Example 5

### Verification that Phagocytosis Stimulating Effect is Caused by Polysaccharides of Hoodia Gordonii and not Due to LPS Contamination

### Enzymatic Hydrolysis of Polysaccharides.

A stock solution of an edible composition comprising a polysaccharide-enriched extract of Hoodia Gordonii (B) of 20 gram/liter was made in acetate buffer (10 mM NaAc+140 mM NaCl in water adjusted with HCl to pH5). A lipopolysaccharide E-coli L4130 (LPS) solution of 13.3 g/l was made. Both solutions were heated for 10 minutes at 40° C. to improve dissolution. Afterwards, 100 μl of a 10× diluted pectinase enzyme-mix Macer8™ (Biocatalysts, UK) in acetate buffer or 100 μl acetate buffer (blank) was added to 500 μl of the heated sample and 400 μl of acetate buffer. All mixtures were treated overnight at 25° C. (slowly shaken) to degrade complex polysaccharides. The positive control samples were chemically hydrolyzed by adding 250 μl 4N HCl to 500 μl of sample, heating for 2 hours at 99° C. and slowly cooling down to RT overnight. The chemical hydrolyzed samples were neutralized with 250 μl 4M NaOH. After centrifugation for 5 minutes at 15.800×g, the amount of saccharides left in the samples was measured according to the method of Bernfeld as described above.

TABLE 2
Degradation of polysaccharides by chemical and Macer8 ™ hydrolysis,
expressed in formation of small saccharides (Eq. glucose mg/g).

| | Blanco buffer- | Chemically | Macer8 ™- |
|---|---|---|---|
| | treated sample | hydrolyzed | treated sample |
| | Eq. Glucose | sample Eq. | Eq. glucose |
| Sample | (mg/g) | glucose (mg/g) | (mg/g) |
| PS-enriched extract | 55.3 | 753.6 | 686.6 |
| of Hoodia Gordonii | | | |
| Lipopolysaccharides | 0 | 267.4 | 0 |
| E-coli L4130 | | | |

Enzymatic hydrolysis of a polysaccharide-enriched extract of Hoodia Gordonii (B) with Macer8™ results in 90.4% hydrolysis of saccharides, compared to chemical hydrolysis (set as 100%). Both acetate blanco buffer-treated LPS and Macer8™ treated LPS samples did not result in hydrolysis of saccharides (see table 2).

Phagocytosis Stimulating Effect of LPS with and without Enzymatic Hydrolysis of Polysaccharides

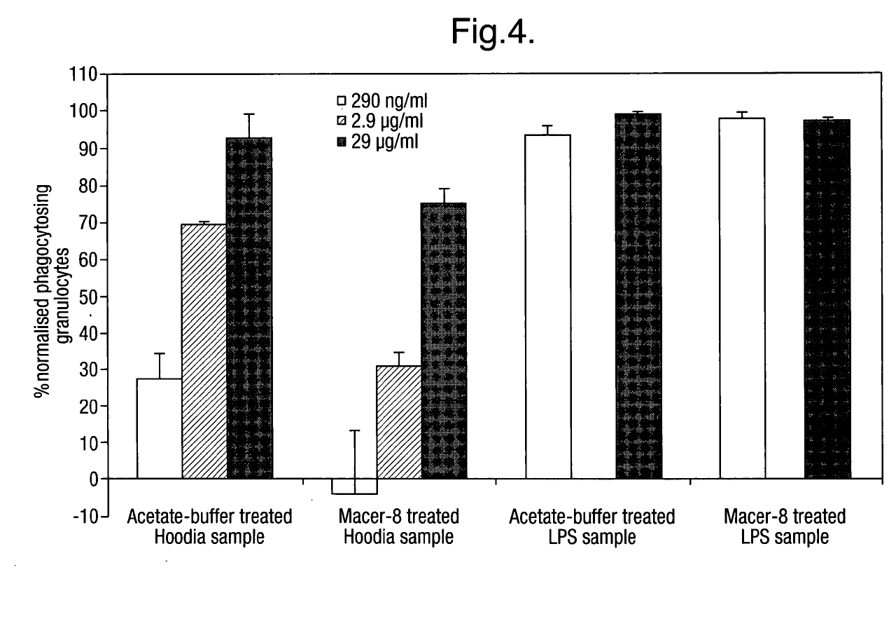

To verify that enzymatic hydrolysis of LPS with Macer8™ does not affect the phagocytosis stimulating activity of LPS, both acetate buffer and Macer8™-treated LPS were tested in the phagocytosis assay at two different concentrations (290 ng/ml and 29 μg/ml). Results (see FIG. 4) represent means and the standard deviation of one experiment with each sample performed in duplicate. FIG. 4 demonstrates the phagocytosis stimulating activity of a polysaccharide-enriched extract of Hoodia Gordonii (B) at a concentration of 0.29-2.9-29 ug/ml and of LPS at a concentration of 290 ng/ml and 29 ug/ml treated with the pectinase enzyme-mix Macer8™ or acetate blanco-buffer. The percentage of phagocytosing granulocytes is reported as percentage of maximal stimulation by LPS, whereby a % normalized phagocytosing granulocytes of >40% can be regarded as an immune stimulating effect.

The results demonstrate that enzymatic hydrolysis of LPS, which did not result in polysaccharide degradation, also did not affect the phagocytosis stimulating ability of LPS.

Phagocytosis Stimulating Effect of a Polysaccharide-Enriched Extract of Hoodia Gordonii with and without Enzymatic Hydrolysis of Polysaccharides

To verify whether polysaccharides in Hoodia Gordonii are responsible for the immunostimulating effect, both acetate blanco buffer and Macer8™ treated polysaccharide-enriched extracts of Hoodia Gordonii (B) were tested in the phagocytosis assay. FIG. 4 shows the effect of polysaccharide-hydrolysis on the phagocytosis activity of blood granulocytes treated with the polysaccharide-enriched Hoodia Gordonii samples. Both acetate-buffer treated samples and the Macer8™ treated Hoodia samples were tested in the phagocytosis assay at three different concentrations (290 ng/ml-2.9 μg/ml-29 μg/ml). Results represent means and the standard deviation of one experiment with each sample performed in duplicate.

The results demonstrate that enzymatic hydrolysis of polysaccharides results in a decrease in the phagocytosis stimulating effect of a polysaccharide-enriched extract of Hoodia Gordonii, verifying that the polysaccharides in Hoodia Gordonii are responsible for the immune stimulating effect.

1. Edible product having an immunostimulating effect, said product comprising immunostimulating polysaccharides obtainable from plants of the Asclepiadoideae subfamily.

2. Edible product according to claim 1, wherein the polysaccharides are obtainable from plants of the tribe Stapelieae.

3. Edible product according to claim 1, wherein the polysaccharides are obtainable from plants of the Hoodia genus.

4. Edible product according to claim 1, wherein the polysaccharides are obtainable from plants of the species Hoodia gordonii.

5. Edible product according to claim 1, wherein the polysaccharides have an average degree of polymerization >3, preferably >5, more preferably >7 to 10.

6. Edible product according to claim 1, wherein the immunostimulating polysaccharides are obtainable by a process comprising the steps of cutting up the plants, extracting the cut material with MeOH:water (90:10% w/w) and preparing a hot-water extract of the insoluble material.

7. Edible product according to claim 6, wherein the hot-water extract is enriched by additional washing steps of the insoluble material.

8. Edible product according to claim 1, in the form of a liquid, such as a soup, a beverage, a spread, a dressing, a dessert or a mayonnaise.

9. Edible product according to claim 1, further comprising one or more steroidal glycosides having the formula 1: 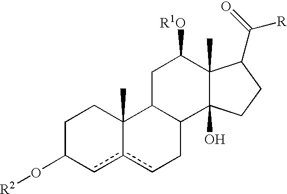
wherein
R=alkyl;
R¹=H, alkyl, tiglyol, benzoyl or any other organic ester group;
R²=H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6.

10. Process for preparing an edible product according to claim 1, comprising the steps of
(a) cutting up the plants,
(b) extracting the cut material with MeOH:water (90:10% w/w),
(c) preparing a hot-water extract of the insoluble material and
(d) adding said extract to a edible product.

11. Process according to claim 10, wherein the hot-water extract is enriched by additional washing steps of the insoluble material.

12. Composition comprising from 0.0001 to 25% by weight of immunostimulating polysaccharides obtainable from obtainable from plants of the Asclepiadoideae subfamily and having an immunostimulating effect, said polysaccharides having an average degree of polymerization >3, preferably >5, more preferably >7 up to 1,000.
